SEQUENCE LISTING (5 sequences)

SEQ ID NO: 1 - RNA, 21 nt, Artificial Sequence
  Description of Artificial Sequence Synthetic primer
aagaucucccuuaagagcaaa

SEQ ID NO: 2 - RNA, 21 nt, Artificial Sequence
  Description of Artificial Sequence Synthetic primer
aagacuucauguguaguugau

SEQ ID NO: 3 - DNA, 20 bp, Artificial Sequence
  Description of Artificial Sequence Synthetic primer
atggttatggtgatcaaagc

SEQ ID NO: 4 - DNA, 20 bp, Artificial Sequence
  Description of Artificial Sequence Synthetic primer
atggcccactacgtgaacca

SEQ ID NO: 5 - DNA, 100 bp, Artificial Sequence
  Description of Artificial Sequence Synthetic primer
agcctcggggaggaggaaggcatcagcgatctcttcgatgcttacgatttggaaaagctcccactggtggaagactttatgtgctcataattatgcttcg

(19) United States
(12) Patent Application Publication
(10) Pub. No.: US 2010/0286228 A1
(43) Pub. Date: Nov. 11, 2010

(54) Method of inhibiting intimal hyperplasia

(75) Inventor: Bruce A. Sullenger, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY

(21) Appl. No.: 11/921,084

(22) PCT Filed: Jun. 1, 2006
(86) PCT No.: PCT/US06/21154

§ 371 Date: Nov. 27, 2007

Related U.S. Application Data
(60) Provisional application No. 60/686,048, filed on Jun. 1, 2005.

(51) Int. Cl. A61K 31/713; C07H 21/02; A61P 35/00; C12N 5/071

(52) U.S. Cl. 514/44 A; 536/24.5; 435/325

(57) ABSTRACT

The present invention relates, in general, to intimal hyperplasia, and, in particular, to a method of inhibiting intimal hyperplasia using siRNA to E2F. The invention further relates to compounds and compositions suitable for use in such a method.

This application claims priority from Provisional Application No. 60/686,048, filed Jun. 1, 2005, the entire content of which is incorporated herein by reference.

### TECHNICAL FIELD

The present invention relates, in general, to intimal hyperplasia, and, in particular, to a method of inhibiting intimal hyperplasia using siRNA to E2F. The invention further relates to compounds and compositions suitable for use in such a method.

### BACKGROUND

One method for specifically inhibiting gene expression is via delivery of short interfering RNAs (siRNAs). Among the approaches for gene inhibition, including anti-sense oligodeoxynucleotides (ODNs) and ribozymes, siRNA is currently the fastest developing approach for gene inhibition, target validation, and therapeutic applications¹. siRNAs appear to be well-suited for therapeutic application. In addition to their highly specific inhibition of a target gene, siRNAs are effective at low concentrations, thus reducing or eliminating the likelihood of toxicity due to non-specific activity. Recently, several proof-of-principle studies have demonstrated the therapeutic potential of siRNAs. These have included treatments for hepatitis^{2,3}, viral infections^{4,5}, macular degeneration⁶, sepsis⁷, tumor growth and invasiveness⁸⁻¹⁰, chronic neuropathic pain¹¹, and serum cholesterol¹². What is evident from these studies is that targeting siRNAs to particular cell types or delivering them locally further reduces the likelihood of detrimental side effects while increasing the efficiency of the siRNA response. Currently, there is an intensive effort to discover methods for chemical modifications of siRNAs that will further facilitate target delivery as well as increase stability of functional siRNAs in vivo^{l2,13}.

These targeted methods for silencing of specific genes with siRNAs make for attractive therapeutic strategies in the treatment of localized pathological intimal hyperplasia. Pathological intimal hyperplasia occurs in venous by-pass grafts and in arteries following injury incurred during by-pass grafting or angioplasty and is in large part due to the proliferation of vascular smooth muscle cells (VSMCs) in the media and their migration into the intima of the treated vessel^{14,15}. Such proliferation is induced by a number of growth stimulatory signals that are activated by vascular injury¹⁶⁻¹⁸. In addition to increased proliferation, apoptosis of cells in the media leading to inflammation and upregulation of chemokines and their receptors has also been shown to play a role in priming this hyperproliferative response^{19,20}. Such abnormal behavior of vascular cells leads to high long-term failure rates of by-pass surgery and angioplasty for treatment of cardiovascular disease. Indeed, despite refinements in surgical procedures, the rate of vein graft failures remains high^{21,22}. These failures often require repeated treatment by surgery or angioplasty and can result in heart attack or amputation of ischemic organs. Accordingly, development of molecular strategies that effectively inhibit such pathogenic cellular processes has been the focus of much research and many clinical trials over the past 20 years.

The E2F family of transcription factors plays a pivotal role in controlling the expression of genes involved in DNA replication, cell cycle progression, and cell fate determination²³⁻²⁸. To date, eight gene products (E2Fs 1-8) comprise the E2F family of proteins and additional isoforms for E2F3 and E2F6 also exist though their functions have not been well characterized²⁹⁻³². Based on sequence homology, the E2F proteins can be divided into three distinct categories. E2Fs1-3 are tightly regulated during the cell cycle and function mostly as activators of transcription²⁶. E2F4 and E2F5 function as transcriptional repressors in concert with pRb family members, p130 and p107³³. E2F6-8 are believed to function as repressors of transcription independent of the pRb family of proteins^{30,31,34}. In addition, evidence from various groups suggests that the activator E2Fs (E2F1-3) have specific functions. This functional specificity is most evident in a role for E2F3 in control of cell proliferation and a role for E2F1 in the induction of apoptosis^{35,36}.

Because E2F activity plays a central role in controlling cell growth and cell fate determination, inhibition of E2F activity promises to be an effective way to block the cellular processes in vascular smooth muscle cells (VSMCs) associated with pathological intimal hyperplasia. Indeed, recent studies by Eckhart et al.⁴⁰ demonstrated that intimal hyperplasia is greatly reduced in damaged arteries in E2F3 knockout mice.

The present invention results from studies designed to test the ability of siRNAs selectively targeting E2Fs, E2F1 and E2F3 to inhibit proliferation and apoptosis of VSMCs in vitro, as well as for their ability to reduce the development of intimal hyperplasia in a mouse bypass graft model. The invention provides a method of inhibiting pathological intimal hyperplasia that occurs, for example, in venous by-pass grafts and in arteries following injury resulting from by-pass grafting or angioplasty.

### SUMMARY OF THE INVENTION

The present invention relates, in general, to intimal hyperplasia, and, in particular, to a method of inhibiting intimal hyperplasia using siRNA to E2F. The invention further relates to compounds and compositions suitable for use in such a method.

Objects and advantages of the present invention will be clear from the description that follows.

### BRIEF DESCRIPTION OF THE DRAWINGS

FIGS. 1A-1D. Effect of siRNAs against E2F1 and E2F3 on E2F-mediated transcriptional activity. (FIG. 1A) NIH3T3 cells were transfected with E2F1-luc reporter plasmid along with an HA-E2F1 expression vector alone or together with synthetic siRNA duplexes against E2F1 (F1-2, F1-3, F1-4, F1-5) or E2F3 (F3-2). (FIG. 1B) NIH3T3 cells were transfected with p68-luc reporter plasmid along with an HA-E2F3 expression vector alone or together with synthetic siRNA duplexes against E2F3 (F3a-2, F3-2, F3-5, F3-6) or E2F1 (F1-3). Luciferase activity was normalized to Renilla activity from three independent experiments. Mouse vena cava vascular smooth muscle cells (VSMCs) were transfected using a lipid base reagent with either a non-specific control siRNA (control) or siRNAs to either: (FIG. 1C) E2F1 (F1-2, F1-3, F1-4, F1-5) or (FIG. 1D) E2F3 (F3-2, F3-5, F3-6). The siRNAs were transfected either alone or together (siE2F3 pool, siE2F1 pool). Nuclear extracts from transfected cells were then resolved on SDS acrylamide gels and assessed for presence of E2F proteins by Western blotting with specific antibodies (top panels). Target specificity for each individual siRNA was assessed by determining the levels of a non-target E2F member (bottom panels).

FIGS. 2A-2C. Lack of different E2Fs can reduce or accelerate growth of VSMCs in vitro. (FIG. 2A) Mouse vena cava VSMCs were transfected with either a non-specific control siRNA (scr) or siRNAs to either E2F1 (F1-3, F1-4, F1-5) alone or along with an E2F1 rescue construct that generated a mutant transcript that was not degraded by its target siRNA, F1-3 (Rescue). Cells were then synchronized at the 01/S boundary by addition of 0.5 μM hydroxy urea (HU). After 21 h cells were released from the HU block and stimulated to reenter the cell cycle by addition of media containing serum and ³H-thymidine. 24 h post serum addition cells were lysed and analyzed for ³H-thymidine incorporation using a scintillation counter. (FIG. 2B) Mouse vena cava VSMCs were transfected with either a non-specific control siRNA (scr) or siRNAs to either E2F3 (F3-2, F3-5, F3-6) alone or along with an E2F3 rescue construct that generated a mutant transcript that was not degraded by its target siRNA, F3-6 (Rescue). (FIG. 2C) VSMCs from vena cavae of WT or E2F4−/− mice transfected as described above.

FIG. 3. Lack of E2F1 can reduce apoptosis of VSMCs in vitro. Mouse vena cava VSMCs were transfected with either a non-specific control siRNA (scr), siRNAs to either E2F1 (F1-3) or E2F3 (F3-2, F3-6), or F1-3 along with the E2F1 rescue construct. 24 h post transfection, cells were treated with 100 μM cisplatin for 30 h. Cells were then fixed and stained for active caspase 3 using a PE-conjugated antibody specific to cleaved caspase 3. Flow cytometric analysis was used to quantitate % PE positive cells.

FIGS. 4A-4D. Uptake of siRNAs in venous grafts in vivo. (FIG. 4A) Schematic of experimental approach for assessing delivery of siRNAs in grafted vessels. (FIG. 4B) Assessment of siRNA stability. Three venous grafts of WT mice were incubated with ³²P-scrambled siRNA for 30 min at 25° C. Following incubation the grafts were washed perfusedly, freeze-thawed twice to break up the tissue, and the siRNA extracted using phenol:chlorophorm. The labeled siRNA was subsequently resolved on a non-denaturing acrylamide gel to assess extent of degradation. (FIG. 4C) Assessment of siRNA uptake. The vena cavae were excised from mice and incubated either at room temperature or on ice in DMEM containing a total of 5 nmoles scrambled siRNA and trace amounts (100,000 cpms) of end-labeled ³²P-scrambled siRNA for 30 minutes. The vessels were then washed profusedly before quantitating uptake of ³²P-scrambled siRNA into the vessels. The % uptake was measured by dividing the amount of ³²P within the vessels by the input (100,000 cpms) ³²P-scrambled siRNA X 100. (FIG. 4D) E2F protein products after siRNA treatment. Extracts of venous grafts previously incubated with either scrambled siRNA (SCR) or siRNAs to E2F1 and E2F3 (siE2Fs) were resolved on SDS-PAGE and proteins subsequently transferred onto PVDF membrane for immunoblotting.

FIGS. 5A-5C. siRNAs against E2F1 and E2F3 reduce intimal hyperplasia in venous by-pass grafts. (FIG. 5A) Photomicrographs showing cross-section from murine vein-graft 28 days after implantation treated with (left) pluronic gel alone (Gel control), (middle) non-specific scrambled siRNA (SCR), and (right) siRNAs against E2F1 and E2F3 (siE2F). The venous VSM intimal hyperplasia in the Gel control and SCR treated 28 day graft is highly cellular and composed of smooth muscle cells interspersed in a connective tissue matrix. The vessel wall of the siE2F 28 day graft is only a few cell layers thick. (Modified Masson trichrome and Verhoeff elastin stain). (FIG. 5A′) 40× magnifications of boxed regions in FIG. 5A showing thickness of intimal layer. (FIG. 5B) Mice treated with either no siRNAs (Gel; pluronic gel control), 5 nmoles of control siRNA (SRC), or 2.5 nmoles each of siRNAs against E2F1 and E2F3 (2′0H siRNA). Area of the intimal layer (Intima) and medial layer (Media) of the vessel are represented. The intimal ratio (area of the intima of the vessel divided by the total area of the vessel) was determined 28 days post-bypass graft. (FIG. 5C) Data in FIG. 5B was plotted as % Inhibition, where the gel control group is set to 100% Inhibition of intimal hyperplasia. Each bar represents an average measurement from 13 mice. Intimal Ratio is reduced by ˜42% after treatment with the siRNAs against the E2Fs; P<0.0001. Intimal thickness is reduced by ˜0.44%, P=0.0003, no significant change in Medial thickness is observed; P=0.8727.

### DETAILED DESCRIPTION OF THE INVENTION

The discovery that small interfering RNAs (siRNAs) can inhibit gene expression in a sequence-specific manner in mammalian cells has raised the possibility of treatments for many pathological conditions using such gene inhibitors in vivo. It is shown in the Example that follows that siRNA to E2F1 and E2F3 can inhibit the proliferation and apoptosis of venous primary smooth muscle cells in culture. Moreover ex vivo delivery of these siRNAs to vein grafts results in silencing of the endogenous E2F genes following surgical implantation of the grafts in the mouse. Importantly, administration of siRNAs specific to these growth-promoting E2Fs significantly reduced intimal hyperplasia in the implanted grafts. These studies establish the therapeutic proof of principal that siRNAs can limit intimal hyperplasia in bypass grafts in animals. Thus the E2F specific siRNAs represent lead compounds that may prove useful for inhibiting this pathological process and graft failure following peripheral and coronary bypass graft surgery in man.

Described herein is the development of siRNAs that act as selective inhibitors of the activator E2Fs. The data presented in the Example that follows show that these inhibitors can be effectively delivered to the target site for therapeutic purposes. Specifically, it is shown that short-term, local delivery of siRNAs targeting the growth promoting E2Fs (E2F1 and E2F3) results in reduced intimal hyperplasia following vein bypass grafting in the mouse. The reduction in intimal hyperplasia correlated with the ability of these siRNA inhibitors to block proliferation and apoptosis of vena cavae VSMCs in culture. It is not surprising, given the dual role of E2F1 in the control of cell proliferation and cell fate, that inhibition of cell proliferation was achieved with siRNAs against either E2F1 or E2F3, while inhibition of DNA-damage induced apoptosis was specific to the siRNA against E2F1. The development of molecular strategies that inhibit both pathological cellular proliferation and apoptosis leading to intimal hyperplasia has been the focus of much research^{37,39,42,43}. Indeed, recent reports have suggested that, in addition to increased proliferation, apoptosis of cells in the media following vascular damage may be involved in priming the hyperproliferative response associated with intimal hyperplasia in vivo¹⁹. Because E2F activity is capable of mediating proliferation of cells as well as apoptosis depending on presence or absence of growth stimulatory signals or in response to DNA damage^{36,44}, inhibition of E2F activity promises to be an effective way to block the cellular processes in VSMCs.

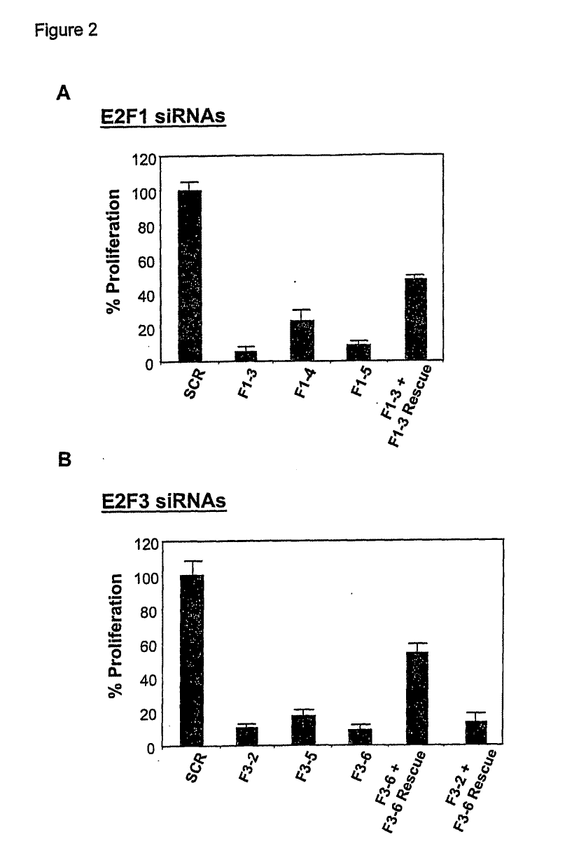

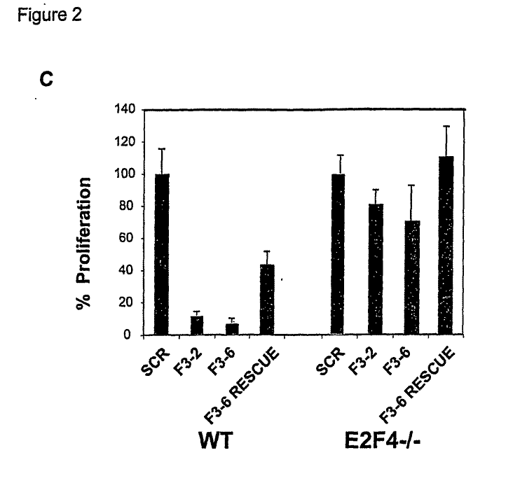

Strikingly, it was observed that the E2F3 siRNAs are only effective at inhibiting VSMC proliferation when E2F4 is present. E2F3 siRNAs are much less effective inhibitors of cell proliferation in VSMCs derived from vena cava of E2F4 knockout mice (FIG. 2C). This result suggests that E2F3 and E2F4 play opposing roles in VSMC proliferation and is consistent with the recent observation that mice lacking E2F4 (a growth arresting E2F) exhibit increased intimal hyperplasia following arterial damage, while mice lacking E2F3 (a growth promoting E2F) show reduced intimal hyperplasia compared to WT control mice⁴⁰. Similarly, mice lacking E2F1 also show a stark reduction in intimal hyperplasia under these experimental conditions. Together, these studies provide strong evidence to suggest that agents such as siRNAs that specifically block only the proliferative and apoptotic functions of the E2Fs would be most effective for limiting restenosis in the clinic. Moreover, they indicate that inhibitory agents that do not distinguish between the various E2F family members, for example ones that inhibit both E2F3 and E2F4 function, will likely be sub-optimal agents for controlling vascular smooth cell proliferation and intimal hyperplasia in the clinic. Consistent with this interpretation, two large randomized phase 3 studies recently demonstrated that a non-selective E2F inhibitor, an E2F DNA decoy, did not significantly impact on intimal hyperplasia and graft failure⁴⁵. Thus one explanation for the lack of clinical efficacy of the E2F DNA decoy, which bears the consensus E2F DNA binding site for all the E2Fs, is that since the DNA decoy can inhibit the activity of both growth stimulating E2Fs and growth repressing E2Fs then its administration may result in a phenotype similar to the one observed when E2F3 siRNAs are not very effective at inhibiting cell proliferation when E2F4 activity is absent.

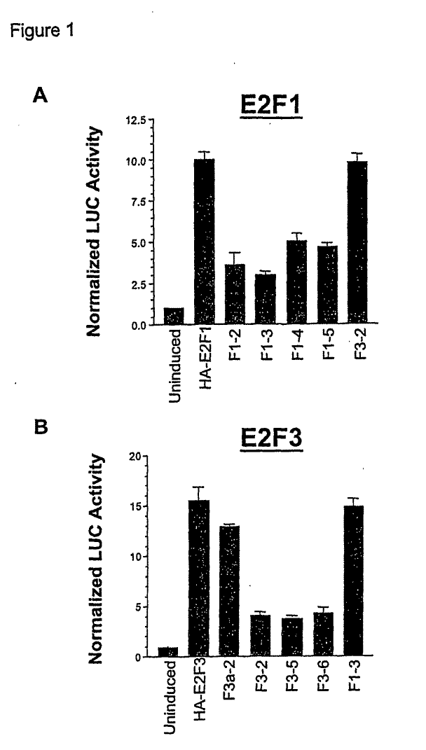

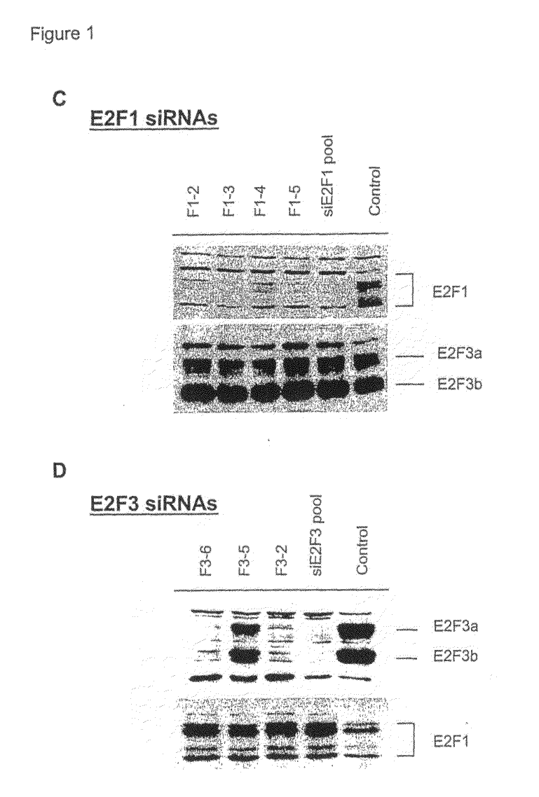

Although technical challenges are still associated with the therapeutic application of siRNAs, such as specificity, cost of synthesis, delivery, and stability, siRNAs are the fastest developing therapeutic approach for gene inhibition. In the therapeutic setting of bypass surgery, many of these hurdles appear to be surmountable. The likelihood of the siRNAs having non-specific toxicity do to non-specific effects on other mRNAs is greatly reduced because the siRNAs are directly and transiently delivered to by-pass grafts ex vivo which should greatly reduce the potential systemic toxicity. To that effect, it has been shown that the siRNAs against the E2Fs are specific for the targeted E2Fs (FIGS. 1C and 1D). Moreover, the delivery of siRNA to grafts ex vivo will substantially the quantity of the siRNA required for treatment and thus reduce the cost of their use in this clinical setting. In addition, currently intensive work is also being performed further increase stability and facilitate cellular delivery and tissue bioavailability of siRNAs^{6,10,12,13}. These improvements in the siRNA technology should also facilitate their use in the setting of cardiac and vascular surgery. Thus, it is anticipated that the clinical utility of siRNAs will be evaluated in the setting of cardiovascular surgery in the near future.

Certain aspects of the invention can be described in greater detail in the non-limiting Example that follows.

### Example

### Experimental Details

Unless otherwise noted, all chemicals were purchased from Sigma-Aldrich Co., all restriction enzymes were obtained from New England BioLabs, Inc. (NEB), and all cell culture products were purchased from Gibco BRL/Life Technologies, a division of Invitrogen Corp.

### Cell Culture

Primary mouse embryonic fibroblasts (MEFs) were maintained at 37° C. and 5% CO₂ in Dulbecco's modified Eagle's medium (DMEM) supplemented with 10% heat inactivated fetal bovine serum. Primary cultures of mouse VSMCs from thoracic aortas were obtained and cultured as described previously^{47,48}. VSMC from aorta of wild type and E2F4−/− mice were maintained in 4-10 Medium.

### Luciferase Assays

NIH/3T3 cells were maintained in DMEM supplemented with 10% fetal bovine serum (Gibco). 5×10⁴ cells/well were seeded in 24-well plates 16 h prior to transfection. Co-transfection of siRNA and reporter plasmids was carried out using Superfect (Qiagen) following the manufacturer's protocol as previously described⁴⁹. Per well, 1 pg of either E2F1-Luc or p68-Luc, 1 ng pRL-TK (Promega), and where indicated, 4 ng of HA-E2F1 or HA-E2F3, and 50 pmoles siRNA duplex were used with a final volume of 360 μl. 24 h post transfection cells were assayed for Luciferase and Renilla expression. Each experiment was performed in triplicate.

### Nuclear Extracts and Westerns

Primary, passage 3, VSMCs from vena cava of wildtype mice were seeded in 60 mm dishes at 50% confluency and transfected twice using Superfect Reagent (Qiagen) with either 1 μM of scramble siRNA (control), 1 μM siRNA against E2F3 (F3-2 alone, F3-5 alone, F3-6 alone, or a combination of F3-1, F3-5, F3-6 (siE2F3 pool), or 1 μM siRNA against E2F1 (F1-5 alone, F1-4 alone, F1-3 alone, F1-2 alone, or a combination of F1-5, -4, -3, -2 (siE2F1 pool)). The first transfection was performed 24 h after seeding the cells while the second transfection was performed 48 h after seeding the cells. This transfection protocol allows for increased transfection efficiencies under these conditions. Cells were allowed to recover for 24 h after the second transfection and then assayed for E2F1 or E2F3 protein expression levels. Nuclear extracts of vena cava VSMCs were prepared as previously described⁴⁹. Extracts were resolved on SDS-PAGE and proteins subsequently transferred onto PVDF membrane for immunoblotting. The following primary antibodies were employed for immunoblotting: anti-E2F3a (SantaCruz, SC-879), anti-E2F1 (SantaCruz, SC-251), anti-E2F2 (SantaCruz, SC-633), and anti-E2F4 (SantaCruz, SC-1082).

### Generation of Mutant E2F Constructs for Use in Rescue Experiments

The siRNA target sequences are as follows: F1-3, AAGAUCUCCCUUAAGAGCAAA and F3-6, AAGACUUCAUGUGUAGUUGAU.

E2F mutants (pcDNA3-HAE2F1 mut and pcDNA3-HAE2F3amut) were generated using standard molecular biology techniques. Briefly, the primers used for the mutagenesis are as follows:

E2F1,
5′-atggttatggtgatcaaagc;
E2F3,
5′-atggcccactacgtgaacca,
5′-agcctcggggaggaggaaggcatcagcgatctcttcgatgcttacga
tttggaaaagctcccactggtggaagactttatgtgctcataattatgct
tcg.

E2F1 mut harbors silent point mutations that render it insensitive to the effect of siRNA F1-3. E2F3amut harbors silent point mutations designed to abrogate targeting by siRNA F3-6.

### Transfection Assays

Primary, passage 3, VSMCs from vena cava of wild type or E2F4−/− mice were seeded in 60 mm dishes at 50% confluency and transfected twice with either 1 μM scrambled siRNA (control), 1 mM siRNA against E2F1 (F1-3, F1-4, or F1-5), 1 μM siRNA against E2F3 (F3-2, F3-5, or F3-6), or 1 μM of F1-3 plus 4 μg of pcDNA3-HAE2F1, or F3-6 plus 4 pg of pcDNA3-HAE2F3amut (Rescue) for 24 hr using Superfect transfection reagent (Qiagen). Cells were also transfected with an siRNA against E2F6 as a control (siE2F6). Following transfection cells were trypsinized and seeded in 12-well plates at ˜20,000 cells/well.

### VSMC Proliferation (DNA Synthesis) Assay

Transfected VSMCs from vena cava of wild type and/or E2F4−/− mice were trypsinized and seeded in 12-well plates at ˜20,000 cells/well. Cells were then forced into a G1/S block by addition of 0.5 μM HU. After 21 hr cells were released from the HU block by addition of media lacking HU and incubated with media containing ³H-thymidine (1 μCi/mL medium) to, monitor DNA synthesis. After 24 hr incubation in the presence of media containing ³H-thymidine cells were washed twice with PBS, washed once with 5% w/v trichloroacetic acid (TCA) (VWR cat# VW3926-2), were collected in 0.5 mL of 0.5N NaOH (VWR cat# VW3221-1) and placed in scintillation vials for measurement of ³H-thymidine incorporation. Data were plotted as % Cell Proliferation where 100% Cell Proliferation is defined by % ³H-thymidine incorporation. ³H-thymidine incorporation for Gel Control was set to 100%.

### VSMC Apoptosis Assay

Transfected VSMCs from vena cava of wild type mice were treated with 4-10 medium alone (WT no cisplatin) or 4-10 medium containing 100 μM cisplatin for 30 h. Cells were then fixed and stained for active caspase 3 using a PE-conjugated antibody specific to cleaved caspase 3 (as specified in manufacturer's protocol) (Pharmingen). Flow cytometric analysis was used to quantitate % PE positive cells as a measure of apoptosis. % Apoptosis is defined by % PE-Positive Cells as measured by Flow cytometric analysis.

In Vivo siRNA Up-Take Assay

The vena cavae from 3 mice per condition were excised as described below and the excised vessels incubated either at room temperature or on ice in DMEM containing a total of 1 μM scrambled siRNA and trace amounts (100,000 cpms) of end-labeled ³²P-scrambled siRNA for 30 minutes. The vessels were then washed profusely with DMEM three times and twice with PBS before quantitating uptake of ³²P-scrambled siRNA into the vessels. Uptake of ³²P-scrambled siRNA was determined by placing the vessels in scintillation fluid and measuring ³²P using a Scintillation Counter. The % Uptake was measured by dividing the amount of ³²P within the vessels by the input (100,000 cpms) ³²P-scrambled siRNA X 100. In addition, following the 30 min incubation at 25° C., the ³²P-scrambled siRNA from one of the vessels was extracted using phenol:chlorophorm and resolved on a non-denaturing acrylamide gel. To assess activity of the siRNAs in vivo, extracts of venous grafts previously incubated with either scrambled siRNA (SCR) or siRNAs to E2F1 and E2F3 (siE2Fs) were resolved on SDS-PAGE and proteins subsequently transferred onto PVDF membrane for immunoblotting. The following primary antibodies were employed for immunoblotting: anti-E2F3a (SantaCruz, SC-879), anti-E2F1 (SantaCruz, SC-251), anti-E2F2 (SantaCruz, SC-633), and anti-E2F4 (SantaCruz, SC-1082).

### In Vivo Venous Mouse By-Pass Graft Model

The venous by-pass graft model in mice was performed as previously described by Zhang and Hagen et al.⁵⁰. Briefly, a 0.8-cm segment of inferior vena cava (IVC) was harvested from a donor mouse and anastomosed to a syngeneic recipient's carotid artery. Prior to transplantation in recipient mouse, the IVC was placed in DMEM solution containing either 5 nmoles of SCR siRNA or a mixture of 2.5 nmoles each of siRNAs against E2F1 and E2F3 for 30 minutes at RT. Meanwhile, in the graft recipient mouse, a 10-mm segment of the left common carotid artery was isolated from surrounding tissues. This segment was occluded proximally and distally with 8-0 nylon sutures, two arteriotomies were created proximally and distally, about 0.8 cm apart, and the vessel was then flushed with saline. End-to-side anastomosis between the IVC and carotid was performed using two fixed sutures at the proximal and distal corners of each arteriotomy and two running sutures, each 180° around the circumference (with 4-6 bites/180°). The carotid segment between the IVC anastomoses was ligated at both ends and cut, thereby stretching the IVC graft. The 8-0 nylon ligatures were then removed and patency of the graft was determined by assessing blood flow through the wall of the satiated graft. The remaining DMEM solution containing the siRNAs was mixed with 30% pleuronic gel (BASF) on ice and transferred to the site of the transplant where the gel was allowed to polymerize. The incision was then closed and the remaining nucleic acid was allowed to diffuse out of the gel into the vein over the next few days. The whole procedure was performed strictly with atraumatic technique with a 96% success rate. Operative time averaged 10 minutes for IVC harvest and 40 minutes for carotid interposition grafting. All operative procedures were performed aseptically, with pentobarbital sodium (50 mg/kg body weight, intraperitoneal) anesthesia, using an operating microscope (WECK Model 029001, zoom 3.6-18, J. K. Hoppl Corporation).

The grafts were harvested four weeks after transplantation. The grafts were exposed through the previous incision and the thoracic cavity was opened. The right atrium was incised and the graft was perfused with PBS through the left ventricle. The grafts were then perfusion-fixed in situ with 10% buffered formalin for 20 minutes at a constant pressure of 100 mm Hg. The grafts ware excised and placed in 10% neutral buffered formalin for 24 hours and then transferred to 70% ethanol until embedding in paraffin. 5 micron serial sections every 0.5 mm with total 4 sections per graft were taken from the middle of the grafts and stained with Mikat, a modified Masson's trichrome and Verhoeff's elastic tissue stain. This staining allowed the identification of collagen as green, elastin as black, cytoplasm as red, and nuclei as black.

Morphometric analysis of tissue sections was performed using images of 40× original magnification, captured using a Nikon camera. Perimeter and area measurements for the lumen, neointima, and media were performed by plainimetry using ImageTool (Version 3.0, UTHSCSA). Neointima was identified by the criss-cross, random-appearing orientation of smooth muscle cells and by the primarily red color imparted by the prevalence of VSMC cytoplasm and relative absence of collagen. Media was recognized by the circular orientation of VSMCs and the primarily green color imparted by collagen. The measurements were used to create concentric circles of area or perimeter equivalent to the measured from the sections, and the radii of these circles were used to calculate the average thickness of each graft layer.

### Statistical Analysis

These results are given as means±SE. Statistical analysis was conducted using a one-way ANOVA. A P-value of 0.05 or less was considered to indicate a significant difference. In addition to a one-way ANOVA, two-tailed unpaired t tests were conducted to compare each treatment group to every other. The siE2F group was significantly different from the SCR and gel control groups, P<0.0001. The SCR group was not significantly different from the gel control group, P>0.05.

### Results

Designing and Evaluating siRNAs Against E2F1 and E2F3

To develop more potent and selective inhibitors of the human and murine growth promoting E2Fs (E2F1 and E2F3) through the use of siRNA technology mouse and human sequences were first aligned and regions of identity were considered for siRNA targeting. Selected sequences were then BLASTed to confirm E2F target-specificity and uniqueness within the human and mouse genomes and approximately six siRNAs for each E2F target were chosen for analysis.

To assess the inhibitory effects of these E2F-specific siRNAs in mouse fibroblasts in culture, transient transfection assays were performed, using lipid-based transfection reagents to measure E2F-mediated transcriptional activation. Specifically, reporter constructs containing a luciferase gene under the control of either the E2F1 or the p68 promoter were co-transfected with the E2F1 (HA-E2F1) or E2F3 (HA-E2F3) expression cassettes, respectively, in the presence or absence of siRNAs against E2F1 or E2F3 (FIG. 1). The inhibitory effect of the various E2F-specific siRNAs on E2F-mediated transactivation was scored by measuring reporter activation following co-transfection of the siRNAs with their E2F counterparts and reporter constructs. It was next demonstrated by western blot analysis that transient delivery of siRNAs against E2F1, (F1-2, F1-3, F1-4, F1-5) (FIG. 1C) or E2F3, (F3-2, F3-5, F3-6) (FIG. 1D) into vena cava VSMC cells specifically reduced the expression of E2F3 and E2F1. Importantly, the siRNAs against E2F3 had no effect on E2F1 protein levels and the siRNAs against E2F1 had no effect on E2F3 protein levels. The efficiency of siRNA transfer was assessed by co-transfection of a non-specific fluorescently labeled siRNA and was determined to be >75% (data not shown). Moreover, analysis of siRNA transfected VSMCs with reduced levels of either E2F1 or E2F3 proteins resulted in significantly decreased proliferation (measured by ³H-thymidine incorporation) of VSMCs in culture (FIGS. 2A and B). This effect was specific to the E2F targeted and could be partially reversed by co-transfection of a gene encoding either a modified E2F1 or a modified E2F3 transcript that was not degraded by the target siRNAs (F1-3 and F3-6 respectively) (Rescue). The reason for the partial reversal is due to lower transfection efficiencies for plasmid DNA vs. siRNAs in primary VSMCs (data not shown). Importantly, the siRNA effect was greater in E2F4+/+ vena Cava VSMCs compared to vena cava VSMCs derived from E2F4−/− littermates (˜90% vs. ˜20% reduction in proliferation, respectively) (FIG. 2C). This observation is consistent with recent findings that reveal the opposing roles of E2F3 and E2F4 in the development of restenosis following arterial damage in vivo⁴⁰. Specifically, it was shown that loss of E2F3 prevents the development of intimal hyperplasia, while loss of E2F4 hastens the progression of intimal hyperplasia following arterial damage. Together, these findings support the notion that “selective” E2F antagonists, such as siRNAs against the growth promoting E2Fs, E2F1 and E2F3, may prove to be more efficacious at inhibiting mammalian proliferation in vitro and in vivo than non-selective inhibitors of the entire family of E2F proteins.

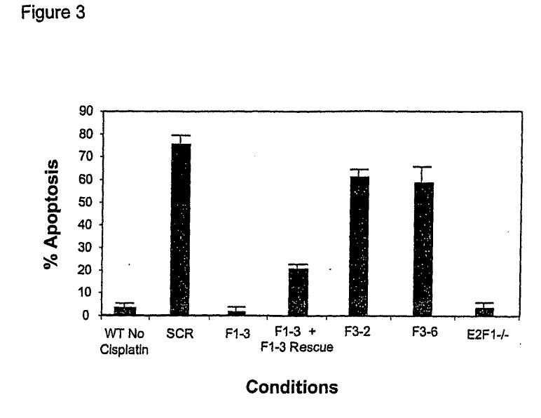

Given the role of E2F1 protein in apoptosis, the effects of inhibition of E2F1 expression on cisplatin-induced apoptosis in VSMCs were assessed. Analysis of siRNA transfected vena cava VSMCs with reduced levels of E2F1 (F1-3) resulted in significantly decreased apoptosis (measured by accumulation of cleaved active caspase 3 using Flow cytometric analysis) of VSMCs in culture (FIG. 3). This effect was specific to the E2F1 siRNA and could be partially reversed by co-transfection of a mutant E2F1 transcript that was not degraded by its target siRNA. Furthermore, siRNAs to E2F3 (F3-2 and F3-6) did not result in decreased apoptosis compared to scramble control (SCR) siRNA.

Uptake of siRNAs in Venous Grafts In Vivo

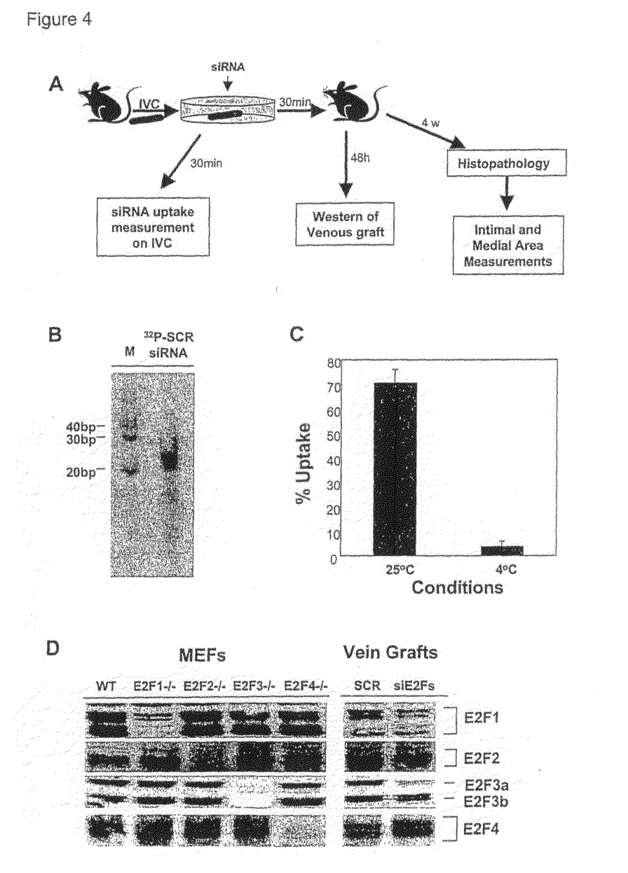

The main obstacle to achieving in vivo gene silencing by RNAi technologies is delivery. First, to assess stability of siRNA in the venous grafts, vena cavae were excised from three mice and incubated with a radiolabeled siRNA (³²P-SCR) for 30 minutes. Then total RNA was isolated from the vessels and intact siRNA was resolved on a non-denaturing PAGE gel (FIGS. 4A and 4B). Next, to determine efficiency of siRNA uptake in the venous grafts, excised vena cavae were incubated with the siRNA ex vivo either at room temperature to allow uptake or on ice to block active transport⁴¹. Grafts were then washed profusely before quantitating uptake of ³²P-siRNA into the vessels. Analysis of uptake revealed that ˜68% of the labeled siRNA had been transported into the venous grafts. In contrast, less than 5% of the input siRNA was associated with the venous grafts incubated on ice (FIG. 4C). It was next demonstrated by western blot analysis that delivery of siRNAs against E2F1 and E2F3 (F1-3 and F3-6 in combination) into mouse venous grafts reduced E2F1 and E2F3 protein expression 48 hours after the grafts had been implanted in mice. Importantly, F1-3 and F3-6 had no effect on the expression of other E2F family members (see E2F2 and E2F4 westerns) (FIG. 4D).

### Reduced Intimal Hyperplasia in Mouse Venous By-Pass Grafts

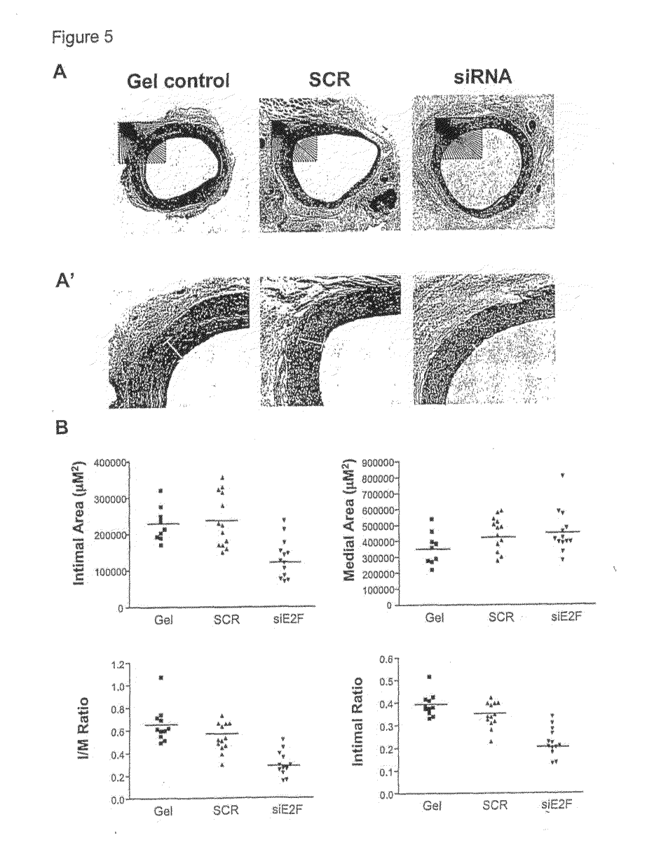

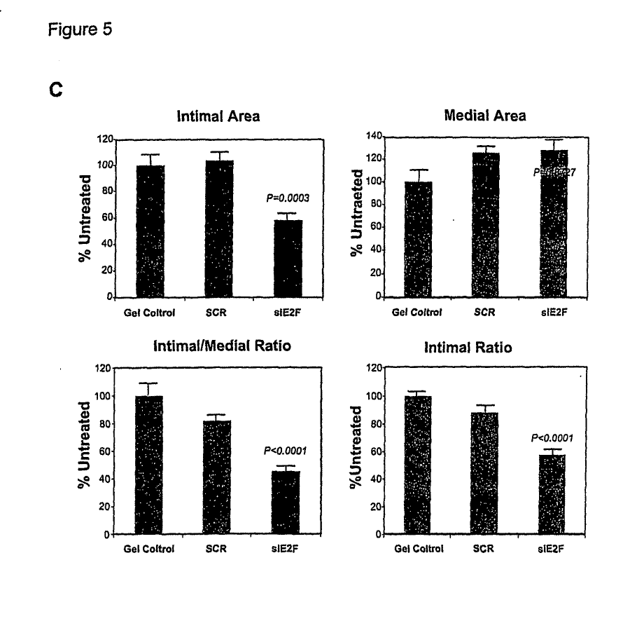

The effects of the siRNAs against the growth promoting E2Fs (E2F1 and E2F3 in combination) on the development of intimal hyperplasia in a mouse model of venous by-pass grafting were next assessed. Briefly, inferior vena cava to carotid artery vein graft procedures were performed on 13 mice per experimental condition. Four weeks post-procedure the grafts were harvested, fixed, sectioned, and stained for analysis. Analysis of the graft sections confirmed that intimal hyperplasia had developed in control animals that received no treatment (Gel control), as well as in animals treated with a scrambled siRNA (SCR) (FIGS. 5A and 5A′). In contrast, treatment of venous grafts with siRNAs against both E2F1 and E2F3 (F1-3 and F3-6, respectively) resulted in a significant decrease in intimal hyperplasia when compared to control samples (FIGS. 5B and 5C). The siRNAs against the E2Fs reduced the Intimal-to-Medial Ratio by ˜42% and ˜57% and the Intimal Ratio by ˜36% and 43% when compared to both SCR control and Gel control groups, P<0.0001 (FIG. 5C, bottom panels). By contrast, the E2F siRNAs had no significant effect on medial area (FIGS. 5B and 5C). In summary, the data indicate that the inhibition of E2F1 and E2F3 protein production by siRNAs significantly reduces the development of intimal hyperplasia in this mouse model of venous bypass grafting.

### REFERENCES

1. Scanlon K J. Anti-genes: siRNA, ribozymes and antisense. Curr Pharm Biotechnol. 5(5), 415-20 (2004).
2. Zender L. et al. Caspase 8 small interfering RNA prevents acute liver failure in mice. Proc. Natl. Acad. Sci. USA 100, 7797-7802 (2003).
3. Song E. et al. RNA interference targeting Fas protects mice from fulminant hepatitis. Nature Med. 9, 347-351 (2003).
4. McCaffrey A. P. et al. Inhibition of hepatitis B virus in mice by RNA interference. Nature Biotechnol. 21, 639-644 (2003).
5. Song E. et al. Sustained small interfering RNA-mediated human immunodeficiency type 1 virus inhibition in primary macrophages. J. Virol. 77, 7174-7181 (2003).
6. Reich S. et al. Small interfering RNA (siRNA) targeting VEGF effectively inhibits ocular neovascularization in a mouse model. Mol. Vis. 9, 210-216 (2003)
7. Sorensen D R, Leirdal M, Sioud M. Gene silencing by systemic delivery of synthetic siRNAs in adult mice. J Mol. Biol. 4, 761-6 (2003).
8. Lapteva N. et al. CXCR4 knockdown by small interfering RNA abrogates breast tumor growth in vivo. Cancer Gene Therapy 12, 84-89 (2005).
9. Liang Z. et al. Silencing of CXCR4 blocks breast cancer metastasis. Cancer Res. 65, 967-71 (2005).
10. Pille' J-Y., et al. Anti-RhoA and anti-RhoC siRNAs Inhibit the Proliferation and Invasiveness of MDA-MB-231 Breast Cancer Cells in vitro and in vivo. Mol. Therapy. 11, 267-274 (2005).
11. Dorn G. et al. siRNA relieves chronic neuropathis pain. Nucleic Acids Res. 32 e49 (2004).
12. Soutscheck J. et al. Therapeutic silencing of an endogenous gene by systemic administration of modified siRNAs. Nature 432, 173-178 (2004).
13. Minakuchi Y. et al. Atellocollagen-mediated synthetic small interfering RNA delivery for effective gene silencing in vitro and in vivo. Nucleic Acids Res. 32, e109 (2004).
14. Clowes A. W., Reidy M. A., and Clowes N. M. (1983). Kinetics of cellular proliferation after arterial injury. I. Smooth muscle growth in the absence of endothelium. Lab Invest. 49, 327-337.
15. Davies, M. G. and Hagen, P. O, Structural and functional consequences of bypass grafting with autologous vein. Cryobiology 31, 63-70 (1994).
16. Wei G L, et al. Temporally and spatially coordinated expression of cell cycle regulatory factors after angioplasty. Circ Res. 80, 418-26 (1997).
17. Kim S, and Iwao H. Stress and vascular responses: mitogen-activated protein kinases and activator protein-1 as promising therapeutic targets of vascular remodeling. J Pharmacol Sci. 91, 177-81 (2003)
18. Chung J K, et al. Expression of cell cycle regulators during smooth muscle cell proliferation after balloon catheter injury of rat artery. J Korean Med. Sci. 19, 327-32 (2004).
19. Pollman M. J., Hall J. L., and Gibbons G. H. Determinants of vascular smooth muscle cell apoptosis after balloon angioplasty injury. Influence of redox state and cell phenotype. Circ. Res. 84, 113-121 (1999).
20. Charo I F and Taubman M B. Chemokines in the pathogenesis of vascular disease. Circ Res. 95, 858-66 (2004).
21. Motwani J D and Topol E J. Aortocoronary vein graft disease: pathogenesis, predisposition, and prevention. Circulation 97, 916-31 (1998).
22. Mehta D, Izzat M B, Bryan A J, and Angelini G D. Towards the prevention of vein graft failure. Int J Cardiol 62, 55-63 (1997).
23. Dyson, N. (1998). The regulation of E2F by pRB-family proteins. Genes Dev. 12, 2245-2262.
24. Nevins, J. R. Toward an understanding of the functional complexity of the E2F and retinoblastoma families. Cell Growth Differ. 9, 585-593 (1998).
25. Ginsberg D. E2F1 pathways to apoptosis. FEBS Lett. 529(1):122-5 (2002):
26. DeGregori, J. The genetics of the E2F family of transcription factors: shared functions and unique roles. Biochemica et Biophysica Acta 1602, 131-150 (2002).
27. Trimarchi J M, and Lees J A. Sibling rivalry in the E2F family. Nat Rev Mol Cell Biol. 3, 11-20. (2002).
28. DeGregori J., Leone G., Miron A., Jakoi L. and Nevins J. R. Distinct roles for E2F proteins in cell growth control and apoptosis. Proc Natl Acad Sci USA 94, 7245-50 (1997).
29. Cartwright P, Muller H, Wagener C, Holm K, Helin K. E2F-6: a novel member of the E2F family is an inhibitor of E2F-dependent transcription. Oncogene 17(5):611-23 (1998).
30. Di Stefano L, Jensen M R, Helin K. E2F7, a novel E2F featuring DP-independent repression of a subset of E2F-regulated genes. EMBO J. 23, 6289-98 (2003).
31. Maiti B, et al. Cloning and characterization of mouse E2F8, a novel mammalian E2F family member capable of blocking cellular proliferation. J Biol. Chem. (2005); [Epub ahead of print]
32. Leone G, et al. Identification of a novel E2F3 product suggests a mechanism for determining specificity of repression by Rb proteins. Mol Cell Biol. 20(10):3626-32 (2000).
33. Gaubatz S, et al. E2F4 and E2F5 play an essential role in pocket protein-mediated G1 control. Mol. Cell. 6(3):729-35 (2000).
34. Trimarchi J M, Fairchild B, Wen J, Lees J A. The E2F6 transcription factor is a component of the mammalian Bmi1-containing polycomb complex. Proc Natl Acad Sci USA. 98(4):1519-24 (2001).
35. DeGregori J., Leone G., Miron A., Jakoi L. and Nevins J. R. Distinct roles for E2F proteins in cell growth control and apoptosis. Proc Natl Acad Sci USA 94, 7245-50 (1997).
36. Wu X, Levine A J. p53 and E2F-1 cooperate to mediate apoptosis. Proc Natl Acad Sci USA. 91(9):3602-6 (1994).
37. Morishita, R. et al. A gene therapy strategy using a transcription factor decoy of the E2F binding site inhibits smooth muscle proliferation in vivo. Proc Natl Acad Sci USA 92, 5855-9 (1995).
38. Mann M J, et al. Genetic engineering of vein grafts resistant to atherosclerosis. Proc Natl Acad Sci USA. 92, 4502-6 (1995).
39. Mann, M J. et al. Ex-vivo gene therapy of human vascular bypass grafts with E2F decoy: the PREVENT single-centre, randomised, controlled trial. Lancet 354, 1493-8 (1999).
40. Eckhart A D, et al. E2F3 and E2F4 Exhibit Opposing Functions in Intimal Hyperplasia. Nature Med.
41. Yakubov L A, Deeva E A, Zarytova V F, Ivanova E M, Ryte A S, Yurchenko L V, Vlassov V V. Mechanism of oligonucleotide uptake by cells: involvement of specific receptors? Proc Natl Acad Sci USA. 86(17):6454-8 (1989).
42. Fulton G. J., et al. Antisense oligonucleotide to proto-oncogene c-myb inhibits the formation of intimal hyperplasia in experimental vein grafts. J. Vasc. Surg. 25, 453-463 (1997).
43. Bai H, et al. Inhibition of intimal hyperplasia after vein grafting by in vivo transfer of human senescent cell-derived inhibitor-1 gene. Gene Ther. 5, 761-9 (1998).
44. Kowalik T F, DeGregori J, Schwarz J K, Nevins J R. E2F1 overexpression in quiescent fibroblasts leads to induction of cellular DNA synthesis and apoptosis. J Virol. 69(4):2491-500 (1995).
45. Mann M. J. and Dzau V. J. Therapeutic applications of transcription factor decoy oligonucleotides. J Clin Invest 106, 1071-1075 (2000).
46. Morishita, R. Perspective in progress of cardiovascular gene therapy. J Pharmacol Sci. 95(1):1-8 (2004).
47. Chen L., Xin X., Eckhart A. D., Yang N., and Faber J. E. Regulation of vascular smooth muscle growth by α1-adrenoreceptor subtypes in vitro and in situ. JBC 270, 30980-30988 (1995).
48. Iaccarino G., Smithwick A. L., Lefkowitz R. J., and Kock W. J. Targeting Gβγ signaling in arterial vascular smooth muscle proliferation: A novel strategy to limit restenosis. Proc. Natl. Acad. Sci. USA 96, 3945-3950 (1999).
49. Giangrande P H, Hallstrom T C, Tunyaplin C, Calame K, Nevins J R. Identification of E-box factor TFE3 as a functional partner for the E2F3 transcription factor. Mol Cell Biol. 23, 3707-20 (2003).
50. Zhang L, Hagen P O, Kisslo J, Peppel K, Freedman N J. Neointimal hyperplasia rapidly reaches steady state in a novel murine vein graft model. J Vasc Surg. 36, 824-32 (2002).

All documents and other information sources cited above are hereby incorporated in their entirety by reference.

What is claimed is:

1. A method of inhibiting intimal hyperplasia in a vein graft in a mammal comprising delivering to said graft an amount of an E2F1- or E2F3-specific interfering RNA sufficient to effect said inhibition.

2. The method according to claim 1 wherein said mammal is a bypass graft surgery patient.

3. The method according to claim 1 wherein said mammal is a peripheral or coronary bypass graft surgery patient.

4. The method according to claim 1 wherein said interfering RNA is E2F1-specific.

5. The method according to claim 1 wherein said interfering RNA is small interfering RNA (siRNA).

6. The method according to claim 1 wherein said interfering RNA is delivered to said graft ex vivo.

7. The method according to claim 1 wherein said interfering RNA does not inhibit E2F4.

8. A method of inhibiting proliferation of mammalian vascular smooth muscle cells comprising delivering to said cells E2F1- or E2F3-specific interfering RNA in an amount sufficient to effect said inhibition.

9. The method according to claim 8 wherein E2F1-specific interfering RNA is delivered.

10. The method according to claim 9 further comprising inhibiting DNA-damage induced apoptosis of said cells.

11. The method according to claim 8 wherein said interfering RNA does not inhibit E2F4.

12. The method according to claim 8 wherein said interfering RNA is siRNA.

13. A method of inhibiting pathological intimal hyperplasia in a patient following injury incurred during by-pass grafting or angioplasty comprising delivering an effective amount of an agent that specifically blocks the proliferative and apoptotic functions of E2Fs.

14. The method according to claim 13 wherein said agent is E2F1- or E2F3-specific siRNA.

15. The method according to claim 13 wherein said injury is incurred during by-pass grafting.

16. The method according to claim 15 wherein said agent is administered to said graft ex vivo.

17. The method according to claim 13 wherein said agent does not inhibit E2F4.

18. An E2F1- or E2F3-specific siRNA.

19. A composition comprising said siRNA according to claim 18 and a carrier.
